(19) United States
(12) Patent Application Publication
(10) Pub. No.: US 2013/0149318 A1
(43) Pub. Date: Jun. 13, 2013

(54) PAINTING THE PIA, ARACHNOID, AND SPINAL CORD PARENCHYMA

(71) Applicant: InVivo Therapeutics Corporation, Cambridge, MA (US)

(72) Inventors: Francis M. Reynolds, Lafayette Hill, PA (US); Robert S. Langer, Newton, MA (US); Jonathan R. Slotkin, Potomac, MD (US); Edward D. Wirth, III, Mountain View, CA (US); Timothy O'Shea, Cambridge, MA (US); Alex Aimetti, Waltham, MA (US)

(73) Assignee: INVIVO THERAPEUTICS CORPORATION, Cambridge, MA (US)

(21) Appl. No.: 13/714,081

(22) Filed: Dec. 13, 2012

Related U.S. Application Data
(60) Provisional application No. 61/570,155, filed on Dec. 13, 2011.

(51) Int. Cl. A61K 47/20

(52) U.S. Cl. CPC A61K 47/20
USPC 424/173.1; 514/772; 514/785; 514/1.1; 424/130.1; 514/44 R; 514/21.9; 514/21.8; 514/169; 514/179; 514/8.1; 514/8.2; 514/20.9; 424/94.61; 424/172.1; 514/282; 514/392; 514/561; 514/330; 514/567; 514/21.1; 424/178.1

(57) ABSTRACT

A PEG based hydrogel and a procedure for its topical application to the surface of the pia mater of the spinal cord that can be used for intrathecal delivery of diverse drug and biomolecular therapies for the treatment of traumatic central nervous system injuries and disorders including spinal cord injury (SCI), multiple sclerosis (MS) and amyotrophic lateral sclerosis (ALS) are provided. This “painting of the pia” with biofunctionalized hydrogel material may be used as a prelude strategy in the therapeutic management of these CNS disorders. The strategy may be designed to create a microenvironment within the damaged regions of the spinal cord that is more conducive to the successful application of subsequent regeneration based treatments such as cell replacement therapies or endogenous regeneration and plasticity stimulation via application of growth factors or gene therapy. Compositions and methods for topical application of the PEG based hydrogel to the arachnoid mater, the intrathecal portions of the spinal nerves, and application directly to the spinal cord parenchyma are also provided.

### CROSS REFERENCE TO RELATED APPLICATION

This application claims the benefit of U.S. provisional application No. 61/570,155, filed Dec. 13, 2011, which is incorporated herein by reference as if fully set forth.

### FIELD OF INVENTION

A Polyethylene glycol (PEG) based hydrogel, its synthesis, and a procedure for its topical application to the surface of tissue.

### BACKGROUND

Disorders and injuries of the spinal cord, the central nervous system (CNS) structure that transmits sensory and motor signals between the brain and the rest of the body, results in debilitating paralysis and a substantial burden of disease for affected individuals. There is a need in the medical community for a clinically effective therapy or surgical intervention that can reverse the permanent disability seen in spinal cord injury (SCI), multiple sclerosis (MS), Amyotrophic lateral sclerosis (ALS), transverse myelitis, and neuromyelitis optica. This disability can include loss of sensation and motor function, loss of control over bowel and bladder function, loss of sexual function, and development of chronic pain. Recovery of lost neurological function and preventing or alleviating chronic pain will likely involve mitigating inhibitory processes unique to CNS and spinal cord pathophysiology as well as activating regeneration and repair mechanisms through endogenous cell populations or cellular transplantation. There are a diverse array of therapies involving the use of small molecules, recombinant proteins, cell transplants and gene therapy that have been investigated pre-clinically that address these goals. While efficacy is often observed in SCI animal models, clinical translation has been hampered by problems associated with localized targeted delivery of the therapies to the human spinal cord. Rationally designed biomaterials can be used to overcome these delivery challenges and provide safe long term local administration to damaged SCI tissue that augments the efficacy and specificity of therapies.

### SUMMARY

In an aspect, the invention relates to a method of treating a patient comprising administering a PEG based hydrogel to a patient in need thereof to at least one site of administration. The at least one site of administration is selected from the group consisting of spinal cord pia mater of the patient, arachnoid mater of the patient, intrathecal portions of spinal nerves of the patient, and directly to spinal cord parenchyma of the patient.

In an aspect, the invention relates to a composition comprising a PEG based hydrogel comprising an aqueous solvent and formed by reaction of a donor and an acceptor via a step growth, base-catalyzed reaction between the donor and the acceptor, the donor having a nucleophilic functional group and the acceptor having an electrophilic functional group.

### BRIEF DESCRIPTION OF THE DRAWINGS

The following detailed description of the preferred embodiment of the present invention will be better understood when read in conjunction with the appended drawing. For the purpose of illustrating the invention, there is shown in the drawing an embodiment. It is understood, however, that the invention is not limited to the precise arrangements and instrumentalities shown. In the drawing:

FIG. 1 illustrates a schematic diagram of a method of treating a patient comprising administering a PEG based hydrogel to a patient in need thereof to at least one site of administration.

FIG. 2 illustrates a schematic diagram of a method of treating a patient comprising administering a PEG based hydrogel to a patient in need thereof to at least one site of administration.

### DETAILED DESCRIPTION OF THE PREFERRED EMBODIMENTS

The words “a” and “one,” as used in the claims and in the corresponding portions of the specification, are defined as including one or more of the referenced item unless specifically stated otherwise. The phrase “at least one” followed by a list of two or more items, such as “A, B, or C,” means any individual one of A, B or C as well as any combination thereof.

Embodiments include compositions comprising a PEG based hydrogel. The PEG based hydrogel may comprise an aqueous solvent and may be formed by reaction of a donor and an acceptor via a step growth, base-catalyzed reaction between the donor and the acceptor. The donor may have a nucleophilic functional group and the acceptor may have an electrophilic functional group.

The aqueous solvent may be an isotonic buffer that has a salt ion concentration modeled on cerebral spinal fluid. The isotonic buffer may have a pH between 7.2-7.3. The isotonic buffer may have an osmolarity between 270-310 mOsm/kg as measured by freezing point depression osmometry. The salt ion concentration may be an artificial cerebral spinal fluid comprising 149 mM sodium chloride (NaCl), 3 mM potassium chloride (KCl), 1.4 mM calcium chloride dihydrate (CaCl₂.2H₂O), 0.8 mM magnesium chloride hexahydrate (MgCl₂.6H₂O), 0.8 mM sodium phosphate dibasic (Na₂HPO₄), and 0.2 mM sodium phosphate monobasic (NaH₂PO₄).

The donor may be a trifunctional thiol polymer. The donor may be ethoxylated trimethylolpropane tri-3-mercaptopropionate. The ethoxylated trimethylolpropane tri-3-mercaptopropionate (ETTMP) may be in a PEG based hydrogel at a concentration of 40 weight percent polymer. The weight percent of ETTMP may be defined per the following equation: [(mass of ETTMP)/(mass of ETTMP+mass of aqueous buffer or water)]*100. The weight percent of polymer in a hydrogel may be defined per the following equation: [(mass of total polymer)/(mass of total polymer+mass of aqueous buffer or water)]*100. The total mass arrived at through the denominator of both equations and used to calculate weight percent may include the mass attributed to other components. For example, the mass of an additional agent, a therapeutic agent, or a bioactive epitope may be included in the denominator. However, in many instances the total mass of other components will be small compared to the weight of polymer and aqueous buffer or water, and the calculated weight fraction may be closely approximated without consideration of the other component mass.

The acceptor may be a bifunctional acrylate polymer. The acceptor may be poly(ethylene glycol) diacrylate. The poly(ethylene glycol) diacrylate may have an average Mn of ˜575 g/mol-1100 g/mol. The poly(ethylene glycol) diacrylate may have an average Mn of ˜575 g/mol. The poly(ethylene glycol) diacrylate may have an average Mn of ˜675 g/mol-725 g/mol. The poly(ethylene glycol) diacrylate may have an average Mn of ˜900 g/mol-1100 g/mol. The poly(ethylene glycol) diacrylate may have an average Mn value within the range ˜575 g/mol-1100 g/mol. The poly(ethylene glycol) diacrylate may have an average Mn value within a range between and including any two values from 575 g/mol-1100 g/mol in one g/mol increments. For example, the poly(ethylene glycol) diacrylate may have an average Mn value within a range between and including 576 g/mole-872 g/mol, or 577-871 g/mol.

The nucleophilic functional group may be a thiol.

The electrophilic functional group may be an acrylate.

In an embodiment, the PEG based hydrogel may include a bioactive epitope in the PEG based hydrogel. The bioactive epitope may be covalently bound to the PEG based hydrogel. The bioactive epitope may include one or more of a peptide, a protein, an antibody, or an aptamer. The peptide may be RGD or IKVAV. The bioactive epitope may be other biomolecules. These peptides or other biomolecules may be incorporated within the gel prepolymer solution. They may have similar functional groups to the other substituents such as acrylates or sulfhydryls resulting in a thioether bond. A peptide may be incorporated as a crosslink (bifunctional acrylate or sulfhydryl) or a pendant group (monofunctional acrylate or sulfhydryl). The PEG based hydrogel may be the same as described above.

In an embodiment, the PEG based hydrogel may include at least one additional agent. The additional agent may be a corticosteroid, methylprednisolone, an anti-inflammatory drug, an anti-CD11d antibody, an angiogenesis promoting growth factor, VEGF, PDGF, decorin, chondroitinase ABC, an anti-Nogo-A antibody, recombinant BA-210 protein, an agent that can alleviate pain, morphine, clonidine, gabapentin, bupivicane, ziconotide, or baclofen.

The additional agent may include one or more therapeutic agent. The concentration of a therapeutic agent in the PEG based hydrogel may be selected to provide a dosage within the range of the clinically recommended dosage of the therapeutic agent. The therapeutic agent may be provided in the PEG based hydrogel. The concentration of a therapeutic agent in the PEG based hydrogel may be 0.05-60 mg in 0.01 ml-5 ml of hydrogel. The amount of a therapeutic agent in 0.01 ml-5 ml of the PEG based hydrogel may be any value within the range between and including 0.05-60 mg. The amount of a therapeutic agent in 0.01 ml-5 ml of the PEG based hydrogel may be any value within the range between and including any two values from 0.05-60 mg in 0.05 mg increments. The amount of a therapeutic agent in 0.01 ml-5 ml of the PEG based hydrogel may be any value within the range between and including any two values from 1-60 mg in 1 mg increments. The concentration of a therapeutic agent in the PEG based hydrogel may be from 0.01 μg/ml up to 12 mg/ml in the hydrogel. The concentration of a therapeutic agent in the PEG based hydrogel may be a value in the range from 0.01 μg/ml up to 12 mg/ml in the hydrogel. The concentration of a therapeutic agent in the PEG based hydrogel may be a value in a range between and including any two concentrations selected from 0.01 μg/ml up to 12 mg/ml in 0.01 μg increments. The concentration of a therapeutic agent in the PEG based hydrogel may be a value in a range between and including any two concentrations selected from 1 μg/ml up to 12 mg/ml in 1 μg increments. In an embodiment, the therapeutic agent may be the anti-inflammatory drug methylprednisolone. The concentration of the methylprednisolone in the PEG based hydrogel may be as set forth above. The concentration of the methylprednisolone in the PEG based hydrogel may be 0.5-60 mg in 0.01 ml-5 ml of hydrogel. The amount of the methylprednisolone in the 0.01 ml-5 ml PEG based hydrogel may be a value in the range 0.5-60 mg. The amount of the methylprednisolone in the 0.01 ml-5 ml PEG based hydrogel may be a value in a range between and including any two values from 0.5-60 mg in 0.5 mg increments. In application, the volume of PEG based hydrogel administered may depend on the individual, the location at which the hydrogel is applied, and the extent of spinal cord to which the therapeutic agent is intended to reach.

The PEG based hydrogel may be formed via the based catalyzed Michael addition reaction between two low molecular weight PEG based polymers with: (1) tri-functional sulfhydryl reactive groups (Ethoxylated Trimethylolpropane Tri-3-mercaptopropionate (ETTMP Thiocure® 1300, Bruno Bock; Mw˜1274 g/mol; viscosity 450 mPas; density=1.15 g/cm3); and (2) bifunctional acrylate reactive groups (Poly(ethylene glycol) diacrylate (PEG-DA) [Sigma Aldrich, #437441 (Average Mn˜575 g/mol), #455008 (Mn˜675-725 g/mol), and #729086 (Mn˜900-1100 g/mol)]. Due to an overall chemical functionality of greater than 2 in the thiol-acrylate polymer system the mixed solution forms an insoluble viscoelastic network when the polymers are combined together in stoichiometric equivalency under slightly basic aqueous conditions. The reaction takes place in an isotonic buffer (pH=7.2-7.3; osmolality ranging between 270-310 mOsm/kg as measured by freezing point depression osmometry) that has a salt ion concentration modeled on cerebral spinal fluid (CSF), referred to as artificial CSF (aCSF). In an embodiment, aCSF contains: 149 mM sodium chloride (NaCl), 3 mM potassium chloride (KCl); 1.4 mM calcium chloride dihydrate (CaCl₂.2H₂O); 0.8 mM magnesium chloride hexahydrate (MgCl₂.6H₂O); 0.8 mM sodium phosphate dibasic (Na₂HPO₄); and 0.2 mM sodium phosphate monobasic (NaH₂PO₄). As this PEG based hydrogel system forms under mild physiologically relevant conditions and requires no catalyst addition to initiate gelation, the system can be safely injected as an in situ curing material. This non-toxic in situ curing property can be beneficial for the pia painting application and other applications described herein.

In an embodiment, three different molecular weight species of PEGDA may be used to fabricate unique hydrogel formulations that may be used for the painting of the pia application. The embodiment pertains to the method of applying to gel to the pia (i.e., painting the pia). However, it may also be implemented to apply the PEG based hydrogel to the other locations. The other locations may be arachnoid mater, intrathecal portions of spinal nerves, or spinal cord parenchyma. Therapeutic release of agents from the PEG based hydrogel may be achieved. Furthermore, with each unique PEGDA polymer species there is a range of hydrogel weight fractions (i.e., the fraction of the weight of the hydrogel that is attributed to the polymer matrix (calculated by [(mass of total polymer in hydrogel)/(mass of total polymer plus mass of aqueous buffer or water)]*100) that can be formulated. The weight fraction of PEGDA can be calculated by substituting “mass of PEGDA” for “mass of total polymer.” The range of hydrogel weight fractions may be: (1) for PEGDA, Mn˜575 g/mol the available weight fraction range is 15 to 40%; (2) for the PEGDA, Mn˜675-725 g/mol the available weight fraction range is 10 to 30%; and finally (3) for the PEGDA, Mn 900-1100 g/mol the available weight fraction range is 10 to 20%. The unique combination of PEGDA species and overall polymer weight fraction produces a hydrogel with unique physical and mechanical properties that can be exploited to tailor the degradation, swelling, stiffness, and molecule release kinetics to the desired application. Formulation of hydrogels within the weight fraction ranges described above may display a characteristic syneresis (shrinking) phenomenon at physiological temperatures. The extent of shrinkage is linearly related to the hydrogel weight fraction, with a lower weight percentage having the greatest syneresis. This syneresis phenomenon is due to favorable polymer thermodynamics at 37° C., and results in contraction of the chains within the hydrogel due to reduced polymer-solvent solubility and interaction. Unlike many other hydrogels developed previously, these formulations will not swell uncontrollably following application to the pial surface. This may prevent undue compression and damage of the fragile spinal cord.

The mechanical properties of the hydrogel may be altered with selection of PEGDA species and weight fraction with the elastic modulus of the various material formulations ranging from 0.05 to 0.2 MPa, which is closely matched to the stiffness of the spinal cord parenchyma. The PEG based hydrogel may also display tailored biodegradability, with complete dissolution of the polymer matrix ranging from approximately 1 week right up to 1 year and any time point in between. The variation in hydrogel degradation rate is conferred by differences in the number of effective crosslinks within the system. Higher weight fraction hydrogels using the small PEGDA (Mn˜575 g/mol) may have the slowest degradation profile while low weight fraction PEGDA (Mn˜900-1100 g/mol) hydrogels may dissolve the fastest. In addition, gel degradation may be tailored based on inclusion of a hydrolytically labile functional group including but not limited to esters, amides, anhydrides, epoxides, carbamates, and ureas.

The release kinetics of additional agent, therapeutic agent, and/or bioactive peptide from the hydrogel matrix may depend on the weight fraction and PEGDA species incorporated. Modification of these parameters produce hydrogel systems with unique effective mesh size and density (related to the crosslink density), which confers altered release kinetics. Hydrogels formed using the PEGDA, Mn˜575 g/mol at a weight fraction ranging from 20-40% have demonstrated an ability to controllably release the small molecule corticosteroid methylprednisolone with first order kinetics over a period of several weeks in vitro. Larger molecules such as chondroitinase ABC (MW=100 kDa), or anti-Nogo-A antibody (MW=130 kDa), can be released in a similar fashion from hydrogels formed using the PEGDA, Mn˜900-1100 g/mol species but diffusion of these larger protein species is obstructed in the smaller molecular weight PEGDA hydrogel. The rho inhibitor BA-210 with a intermediate molecular weight of approximately 26 kDa may be released from hydrogels with a PEGDA size of Mn˜675-725 g/mol or Mn˜900-1100 g/mol. The barrier/exclusion of the larger molecular weight species permitted by the smaller PEGDA hydrogels may be exploited in the current application as a possible secondary layer on top of the original painted structure in order to control the directionality of diffusion.

The following describes formulation of an embodiment of the PEG based hydrogel. To formulate the hydrogel, two individual polymer precursors may be first purified by flash chromatography with activated alumina basic as the stationary phase in order to remove polymerization inhibiting storage agents such as monomethyl ether of hydroquinone (MEHQ) or butylated hydroxytoluene (BHT). Following purification the individual polymers may be dissolved in the aCSF buffer at appropriate concentrations. The ETTMP 1300 solution may be prepared at a concentration of 40 weight percent polymer (i.e., 1.725 mL of buffer for every 1 mL of ETTMP 1300 polymer). The 40 weight percent ETTMP 1300 solution is preferred for fabricating any of the hydrogel formulations described in this patent, but is not the only embodiment herein. The concentration of the PEGDA solution may be prepared such that two conditions are met: (i) the overall polymer fraction of the mixture of the PEGDA and ETTMP 1300 solutions totals the specified value; and (ii) the PEGDA solution contains a sufficient fraction of PEGDA such that the stoichiometry of the acrylate and thiol functional groups is equal. Once the polymers have been fully dissolved in the aCSF, the solutions may be transferred to a sterile biosafety cabinet where they are sterile filtered twice using 0.8/02 μm and 0.1 μm syringe filters and then aliquoted into sterile 1.5 mL (11 mm) serum vials which are then crimped with a sterile silicone septum. Alternatively, neat polymers may be filtered under sterile conditions and packaged in 1.5-5.0 mL serum vials. The vials may be packaged together. An embodiment includes a kit including the neat polymers package. A double barreled syringe may be preloaded with the appropriate amount of buffer in each barrel. The solutions may then be injected into the respective serum vials to solubilize the polymer which is then subsequently drawn back up into the double barrel syringe. An appropriate mixing chamber and/or tip (spray, sheet, or stream delivery) may then be placed on the double barreled syringe. Various diameter syringes can be used to precisely tune the ratio at which the solutions are to be combined. The kit may include a double barrel syringe loaded with the polymers.

The polymer solutions may be stored in either a room temperature or 4° C. environment away from sources of light until use.

In an embodiment, the serum vials of polymer precursor solutions may be loaded into individual chambers of a double barreled syringe. A reciprocal screw shaped mixing chamber at the front of the syringe is used to combine the two solutions and specific differences in the diameter of the two syringe chambers is used to ensure the appropriate mixing ratio of the two polymers is produced. The combined hydrogel solution will initially appear cloudy following the mixing of the two individual precursor solutions but will start to become more transparent as gelation proceeds. The final viscoelastic hydrogel that is formed at the completion of the reaction is transparent. The combined solution using the aCSF buffer at pH=7.2 as the aqueous solvent phase may form a hydrogel within approximately 2-10 minutes post mixing. However, the specific time of gelation is dependent on the PEGDA species and overall weight fraction selected. Increasing the pH of the aCSF buffer may increase the rate of the thiol-acrylate reaction and result in a more quickly forming hydrogel product. This embodiment was contemplated primarily for a method of applying to gel to the pia (i.e., painting the pia). However, it is not limited to painting the pia, and methods of applying hydrogel to other sites are contemplated. The other sites may include arachnoid mater, intrathecal portions of spinal nerves, or directly to the spinal cord parenchyma. Through the method, it may be possible to achieve desired therapeutic release of therapeutic agents or additional agents included PEG based hydrogel.

Embodiments herein include methods of treating a patient by administering a PEG based hydrogel to a patient in need thereof to at least one site of administration. The PEG based hydrogel may be any PEG based hydrogel. The PEG based hydrogel may be a PEG based hydrogel described herein. The PEG based hydrogel may be a PEG based hydrogel described in US 2010-0196481 (the pre-grant publication of U.S. Ser. No. 12/567,589, filed Sep. 25, 2009), which is incorporated herein by reference as if fully set forth. The at least one site of administration may include the spinal cord pia mater of the patient, arachnoid mater of the patient, intrathecal portions of spinal nerves of the patient, and directly to spinal cord parenchyma of the patient. Administering may include topical application of the PEG based hydrogel to the surface of the pia mater, the arachnoid mater, the intrathecal portions of the spinal nerves, or the spinal parenchyma. The PEG based hydrogel may include a bioactive peptide or additional agent. The additional agent may be a therapeutic agent. The method may thereby include delivery of diverse drug and biomolecular therapies for the treatment of traumatic central nervous system injuries and disorders. The method may include treating spinal cord injury (SCI), multiple sclerosis (MS), and/or amyotrophic lateral sclerosis (ALS). The patient may be human. The patient may be non-human. The patient may be an SCI patient, an MS patient, or a ALS patient. The patient may have another type of injury, disease, or disorder. The method of treating with a PEG based hydrogel, which may be bifunctionalized, may be used as a prelude strategy in the therapeutic management of these CNS disorders. The strategy may be designed to create a microenvironment within the damaged regions of the spinal cord that is more conducive to the successful application of subsequent regeneration based treatments such as cell replacement therapies or endogenous regeneration and plasticity stimulation via application of growth factors or gene therapy. Accordingly, the method may include one or more additional steps of delivering cell replacement therapies, endogenous regeneration, or plasticity stimulation via application of growth factors or gene therapy. The agents for these steps may be included in the PEG based hydrogel or administered separately.

The method may include applying at least one additional agent at the at least one site of administration. The at least one additional agent may be a corticosteroid, methylprednisolone, an anti-inflammatory drug, an anti-CD11d antibody, an angiogenesis promoting growth factor, VEGF, PDGF, decorin, chondroitinase ABC, an anti-Nogo-A antibody, recombinant BA-210 protein, an agent that can alleviate pain, morphine, clonidine, gabapentin, bupivicane, ziconotide, or baclofen. The concentration of one of the at least one additional agents may be any that achieves a therapeutic affect. The concentration of one of the additional agents in a PEG based hydrogel may be selected from the following: methylprednisolone (0.1-20 mg ml-1), an anti-CD11d antibody (0.0001-0.1 mg ml-1), VEGF (0.001-5 mg ml-1), PDGF (0.001-5 mg ml-1), decorin (0.001-5 mg mL-1), chondroitinase ABC (0.0001-1 mg ml-1), an anti-Nogo-A antibody (0.0001-0.1 mg ml-1), recombinant BA-210 protein (0.001-5 mg ml-1), an agent that can alleviate pain (0.1-200 mg ml-1) where the agent that can alleviate pain is morphine, clonidine, gabapentin, bupivicane, ziconotide, or baclofen. The step of applying the at least one additional agent may occur at one of before, during, or after the step of applying the PEG based hydrogel. One or more of the additional agents may be within the PEG based hydrogel.

The PEG based hydrogel in a method herein may be any PEG based hydrogel. The PEG based hydrogel in a method herein may be obtained through any step-growth chemical reaction between two polymers where the sum of their functionality is greater than or equal to 5. Examples of chemical reactions include, but are not limited to, base-catalyzed Michael-type addition, photoinitiated thiol-ene, 1,3-dipolar cycloaddition between functional groups such as an azide and alkyne, strain-promoted azide-alkyne Cu-free click chemistry, or the reaction between an activated carboxylic acid and an amine. Examples of covalent bonds that are can result from these reactions are thioethers, amides, or 1,2,3-triazoles.

In an embodiment, a hydrogel system herein (including the PEG based hydrogel) may be used to deliver compounds/biomolecules that are intended to achieve one or more of the following: (1) mitigate inflammation and the innate immune response as well as prevent up-regulated signaling of pro-inflammatory cytokines; (2) re-establishment of vascular perfusion in undamaged penumbra tissue within the spinal cord via augmented angiogenesis; and (3) disrupt or alleviate extracellular matrix inhibitors derived from myelin debris and activated glial populations. As non-limiting examples, the PEG based hydrogel in a method or composition herein may include at least one of methylprednisolone to modulate inflammation, VEGF to promote angiogenesis, or chondroitinase ABC to disrupt or prevent ECM matrix inhibitors that are present during gliosis.

To achieve these three treatment goals a variety of commercially available and clinically tested molecules can be loaded into the PEG based hydrogel. Inflammation modulation can be achieved using anti-inflammatory small molecules and corticosteroids. Clinically, mitigating neuroinflammation is a standard approach taken to help prevent destruction of tissue in the spinal cord in instances of SCI and MS. Methylprednisolone, a corticosteroid which reduces the migration of leukocytes and vascular permeability during inflammation has demonstrated beneficial outcomes for patients with SCI when administered in the early acute stages of SCI. However, systemic administration of the steroid presents such significant auxiliary challenges for trauma management that the initial clinical excitement surrounding this drug has been curtailed. In light of this reduced clinical uptake, the hydrogel system described herein may be used to controllably deliver methylprednisolone and other drugs locally at the site of injury to overcome the inefficiencies and bystander effects of systemic delivery. The disruption of a diffuse vascular supply following traumatic damage to the spinal cord also creates an under-perfused penumbra region of undamaged tissue around spinal cord lesions, with the cells contained here eventually undergoing ischemic death in the absence of an intervention. To avoid this additional tissue damage the painting of a hydrogel to the pial surface of the spinal cord containing cocktails of recombinant growth factors such as vascular endothelial growth factor (VEGF) and platelet-derived growth factor (PDGF) can be used to promote local angiogenesis and rescue cord tissue through a more rapid initiation and maturation of new blood vessels. This may also be done when administering the PEG based hydrogel to the arachnoid mater, intrathecal portions of spinal nerves, or directly to spinal cord parenchyma. Any one or more of the above agents may be loaded into the PEG based hydrogel in embodiments herein.

Extracellular extrinsic inhibition of SCI regeneration is brought about by the glial response to the initial CNS insult. There are two subpopulations within this category of regeneration inhibitors: (i) myelin derived proteins such as Nogo A, MAG, ephrins etc., which are expressed by oligodendroglia and present in the debris of demyelinated axons; and (ii) a prominent gliosis composed of reactive astrocytes synthesizing chondroitin sulfate proteoglycans (CSPGs) induced through an injury specific cellular phenotype. The extracellular inhibitory species interact with receptors on intact and damaged axons and initiate intracellular signaling cascades involving the GTPase RhoA and other kinases, which provoke destructive remodeling of the actin and microtubule cytoskeleton resulting in dystrophic axonal retraction bulbs and a discontinuation of axon growth kinetics. Specific drugs and recombinant proteins that act on constituents of extrinsic inhibition have been identified and include an anti-Nogo-A antibody; the rho pathway inhibitor, BA-210 (Cethrin); and chondroitinase ABC, to degrade CSPGs. For these molecules achieving localized delivery of therapeutic dosages, long-term stability of the compound and traversing the blood brain barrier have been obstacles that can be alleviated by the pia painted hydrogel system. One or more drug and/or one or more recombinant protein that acts on constituents of extrinsic inhibition may be loaded into the PEG based hydrogel in method or composition embodiments herein.

Surgical Application of the PEG Based Hydrogel

The methods herein, including administering hydrogel to the pia, arachnoid mater, intrathecal portions of spinal nerves, or directly to spinal cord parenchyma, using biofunctionalized hydrogel material may be applied as a prelude strategy in the therapeutic management of these CNS disorders and are designed to create a microenvironment within the damaged regions of the spinal cord that are more conducive to the successful application of subsequent regeneration based treatments.

To achieve this outcome the PEG based hydrogel may be used to deliver at least one of the following:
Corticosteroids such as methylprednisolone to mitigate inflammation;
Anti-inflammatory drugs (such as Anti-CD11d antibody to block entry of neutrophils; Saville et al., J. Neuroimmunol. 2004, which is incorporated herein by reference as if fully set forth);
Angiogenesis promoting growth factors such as VEGF and PDGF;
Decorin to prevent formation of scar tissue components such as chondroitin sulfate proteoglycans;
Chondroitinase ABC to degrade the chondroitin sulphate proteoglycans present within the gliotic scarring around the spinal cord injury cavity;
Anti-Nogo-A antibody to neutralize the myelin-associated neurite growth inhibitor Nogo A;
Recombinant BA-210 protein, which is an inhibitor of the Rho pathway, a common signaling pathway used by extrinsic inhibitors to provoke destructive remodeling of the actin and microtubule cytoskeleton;
Molecules that can alleviate pain, such as morphine, clonidine, gabapentin, bupivicane, ziconotide;
Baclofen to treat spasticity; or
Neurotrophin-3 (NT-3) or Brain-derived neurotrophic factor (BDNF) to promote axon regeneration.

The PEG based hydrogel (which may include any agent described herein) may be applied to the surface of the pia, arachnoid, spinal cord and/or intrathecal portion of the spinal nerves using a topical application procedure. The administration to these sites may be by way of application of the PEG based hydrogel polymer precursors to the site. The administration to these sites may be by way of application of a pre-formed PEG based hydrogel to the site. The method can be performed via several possible methods. For example, in acute spinal cord injury, the hydrogel could be applied during a decompression/stabilization surgery. Decompression surgery typically entails a laminotomy or laminectomy at the injured spine level(s). This exposes the ligamentum flavum or the dura mater overlying the injured spinal segment(s). In these cases, the dura will be opened and the hydrogel will be applied directly to the arachnoid and/or pia mater overlying the spinal cord, and/or to the spinal nerves. In some cases the pia and arachnoid may have been disrupted due to the prior trauma, so in these cases the hydrogel could be applied directly to the spinal cord parenchyma. This procedure could also be performed during a surgery dedicated to hydrogel application in patients who do not undergo decompression/stabilization surgery and/or in patients with chronic spinal cord injuries.

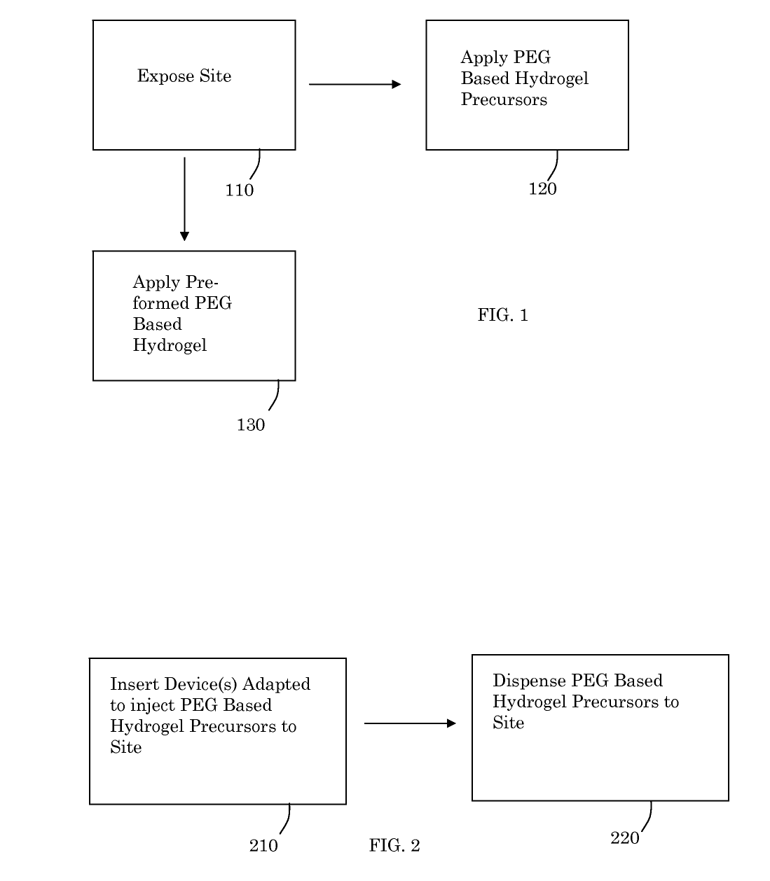

FIGS. 1 and 2 provide non-limiting illustrations of options for the method of treating a patient by administering a PEG based hydrogel to a patient in need thereof to at least one site of administration. As illustrated in FIG. 1, the method may include a step 110 of exposing the site of administration. Any step of exposing may be utilized. The step 110 of exposing may include surgically exposing the site of administration. The step 110 of exposing may include clearing a site of injury to expose the site of administration. The method may also include at least one of: step 120 of applying PEG based hydrogel polymer precursors at the site of administration or step 130 of applying preformed PEG based hydrogel. As illustrated in FIG. 2, the method may include a step 210 of inserting a device(s) adapted to inject PEG based polymer precursors to the site of administration. The device may be a hypodermic needle. The hypodermic needle may be attached to a syringe. The may also include a step 220 of dispensing the PEG based polymer precursors to the site of administration. Dispensing may be accomplished by ejecting polymer precursor(s) from the syringe and through the hypodermic needle. The method may include applying at least one additional agent at the at least one site of administration. The step of applying at least one additional agent may include including the at least one additional agent in the pre-formed PEG based hydrogel or in one or more of the PEG based polymer precursor solutions.

The PEG based hydrogel (which may include any agent described herein) could also be applied to the arachnoid, pia, spinal nerves, and/or spinal cord using minimal access spine surgery, image-guided percutaneous injection, or delivery via an endoscope that is introduced into and advanced through the intrathecal space.

The PEG based hydrogel may be used to deliver one or more of the agents noted above. These agent(s) may be applied in a single application of PEG based hydrogel to the site or via multiple PEG based hydrogel “stripes.” Multiple stripes would facilitate application of several agents during a single procedure, each of which would have a unique time-release duration that is most appropriate for that agent. This embodiment highlights the versatility of using the PEG based hydrogel as a drug release carrier. By applying multiple “stripes” multi-modal release profiles of agents can be achieved for unique therapeutics tailored for a specific application/indication.

One non-limiting example, would be an initial, rapid release of methylprednisolone in an acute spinal cord injury setting (<10 days post injury), followed by a delayed, more sustained release of neurotrophin-3 (NT-3) or chondroitinase ABC (chABC) to promote axon growth and regeneration or prevent gliosis, respectively.

### EMBODIMENT LIST

The following list includes particular embodiments. The list, however, is not limiting and does not exclude alternate embodiments otherwise described or as would be appreciated by one of ordinary skill in the art.

1. A method of treating a patient comprising:

administering a PEG based hydrogel to a patient in need thereof to at least one site of administration, the at least one site of administration selected from the group consisting of spinal cord pia mater of the patient, arachnoid mater of the patient, intrathecal portions of spinal nerves of the patient, and directly to spinal cord parenchyma of the patient.

2. The method of embodiment 1, wherein the step of administering includes applying a composition comprising precursors of the PEG based hydrogel at the at least one site of administration and the precursors react to form the PEG based hydrogel in situ.

3. The method of embodiment 2, wherein the precursors include a donor and an acceptor and the reaction to form the PEG based hydrogel is a step growth, base-catalyzed reaction between the donor and the acceptor, the donor having a nucleophilic functional group and the acceptor having an electrophilic functional group.

4. The method of any one or more of embodiments 2-3, wherein the nucleophilic functional group is a thiol and the electrophilic functional group is an acrylate.

5. The method of any one or more of embodiments 2-4, wherein the donor is a trifunctional thiol polymer and the acceptor is a bifunctional acrylate polymer.

6. The method of any one or more of embodiments 2-5, wherein the donor is ethoxylated trimethylolpropane tri-3-mercaptopropionate and the acceptor is poly(ethylene glycol) diacrylate.

7. The method of embodiment 6, wherein the ethoxylated trimethylolpropane tri-3-mercaptopropionate is added at a concentration of 40 weight percent polymer.

8. The method of any one or more of embodiments 6-7, wherein the poly(ethylene glycol) diacrylate has an average Mn of ˜575 g/mol-1100 g/mol.

9. The method of any one or more of embodiments 6-7, wherein the poly(ethylene glycol) diacrylate has an average Mn of ˜575 g/mol.

10. The method of any one or more of embodiments 6-7, wherein the poly(ethylene glycol) diacrylate has an average Mn of ˜675 g/mol-725 g/mol.

11. The method of any one or more of embodiments 6-7, wherein the poly(ethylene glycol) diacrylate has an average Mn of ˜900 g/mol-1100 g/mol.

12. The method of any one or more of the preceding embodiments, wherein the PEG based hydrogel includes a bioactive epitope and optionally wherein the PEG based hydrogel is covalently modified with the at least one bioactive epitope.

13. The method of embodiment 12, wherein the at least one bioactive epitope includes one or more of a peptide, a protein, an antibody, or an aptamer.

14. The method of embodiment 13, wherein the peptide is selected from the group consisting of RGD and IKVAV.

15. The method of any one or more of embodiments 2-14, wherein the step of forming the PEG based hydrogel occurs in an isotonic buffer that has a salt ion concentration modeled on cerebral spinal fluid.

16. The method of embodiment 15, wherein the isotonic buffer has a pH between 7.2-7.3.

17. The method of embodiment of any one or more of embodiments 15-16, wherein the isotonic buffer has an osmolarity between 270-310 mOsm/kg as measured by freezing point depression osmometry.

18. The method of embodiment 15, wherein the salt ion concentration is artificial cerebral spinal fluid comprising 149 mM sodium chloride (NaCl), 3 mM potassium chloride (KCl), 1.4 mM calcium chloride dihydrate (CaCl₂.2H₂O), 0.8 mM magnesium chloride hexahydrate (MgCl₂.6H₂O), 0.8 mM sodium phosphate dibasic (Na₂HPO₄), and 0.2 mM sodium phosphate monobasic (NaH₂PO₄).

19. The method of any one or more of embodiments 2-18, wherein the composition includes at least one additional agent.

20. The method of embodiment 19, wherein the at least one additional agent is selected from the group consisting of therapeutic agents, a corticosteroid, methylprednisolone, an anti-inflammatory drug, an anti-CD11d antibody, an angiogenesis promoting growth factor, VEGF, PDGF, decorin, chondroitinase ABC, an anti-Nogo-A antibody, recombinant BA-210 protein, an agent that can alleviate pain, morphine, clonidine, gabapentin, bupivicane, ziconotide, and baclofen.

21. The method of any one or more of embodiments 1-18 further comprising applying at least one additional agent at the at least one site of administration.

22. The method of embodiment 21, wherein the at least one additional agent is selected from the group consisting of therapeutic agents, a corticosteroid, methylprednisolone, an anti-inflammatory drug, an anti-CD11d antibody, an angiogenesis promoting growth factor, VEGF, PDGF, decorin, chondroitinase ABC, an anti-Nogo-A antibody, recombinant BA-210 protein, an agent that can alleviate pain, morphine, clonidine, gabapentin, bupivicane, ziconotide, and baclofen.

23. The method of any one or more of embodiments 21-22, wherein the step of applying the at least one additional agent occurs at one or more of before, during, or after the step of applying the composition.

24. A composition comprising a PEG based hydrogel comprising an aqueous solvent and formed by reaction of a donor and an acceptor via a step growth, base-catalyzed reaction between the donor and the acceptor, the donor having a nucleophilic functional group and the acceptor having an electrophilic functional group.

25. The composition of embodiment 24, wherein the nucleophilic functional group is a thiol and the electrophilic functional group is an acrylate.

26. The composition of embodiment 24, wherein the donor is a trifunctional thiol polymer and the acceptor is a bifunctional acrylate polymer.

27. The composition of embodiment 24, wherein the donor is ethoxylated trimethylolpropane tri-3-mercaptopropionate and the acceptor is poly(ethylene glycol) diacrylate.

28. The composition of embodiment 27, wherein the ethoxylated trimethylolpropane tri-3-mercaptopropionate is at a concentration of 40 weight percent polymer.

29. The composition of embodiment 27, wherein the poly(ethylene glycol) diacrylate has an average Mn of ˜575 g/mol-1100 g/mol.

30. The composition of embodiment 27, wherein the poly(ethylene glycol) diacrylate has an average Mn of ˜575 g/mol.

31. The composition of embodiment 27, wherein the poly(ethylene glycol) diacrylate has an average Mn of ˜675 g/mol-725 g/mol.

32. The composition of embodiment 27, wherein the poly(ethylene glycol) diacrylate has an average Mn of ˜900 g/mol-1100 g/mol.

33. The composition of any one or more of embodiments 24-32 further comprising at least one bioactive epitope, wherein the at least one bioactive epitope is optionally covalently bound to the PEG based hydrogel.

34. The composition of embodiment 33, wherein the at least one bioactive epitope includes one or more of a peptide, a protein, an antibody, or an aptamer.

35. The composition of embodiment 34, wherein the peptide is selected from the group consisting of RGD and IKVAV.

36. The composition of any one or more of embodiments 24-35, wherein the aqueous solvent is an isotonic buffer that has a salt ion concentration modeled on cerebral spinal fluid.

37. The composition of embodiment 36, wherein the isotonic buffer has a pH between 7.2-7.3.

38. The composition of any one or more of embodiments 36-37, wherein the isotonic buffer has an osmolarity between 270-310 mOsm/kg as measured by freezing point depression osmometry.

39. The composition of embodiment 36, wherein the salt ion concentration is artificial cerebral spinal fluid comprising 149 mM sodium chloride (NaCl), 3 mM potassium chloride (KCl), 1.4 mM calcium chloride dihydrate (CaCl₂.2H₂O), 0.8 mM magnesium chloride hexahydrate (MgCl₂.6H₂O), 0.8 mM sodium phosphate dibasic (Na₂HPO₄), and 0.2 mM sodium phosphate monobasic (NaH₂PO₄).

40. The composition of any one or more of embodiments 24-39, wherein the composition includes at least one additional agent.

41. The composition of embodiment 40, wherein the at least one additional agent is selected from the group consisting of therapeutic agents, a corticosteroid, methylprednisolone, an anti-inflammatory drug, an anti-CD11d antibody, an angiogenesis promoting growth factor, VEGF, PDGF, decorin, chondroitinase ABC, an anti-Nogo-A antibody, recombinant BA-210 protein, an agent that can alleviate pain, morphine, clonidine, gabapentin, bupivicane, ziconotide, and baclofen.

42. A method of treating a patient comprising:

administering the PEG based hydrogel of any one or more of embodiments 24-41 to a patient in need thereof to at least one site of administration, the at least one site of administration selected from the group consisting of spinal cord pia mater of the patient, arachnoid mater of the patient, intrathecal portions of spinal nerves of the patient, and directly to spinal cord parenchyma of the patient.

Further embodiments herein may be formed by supplementing an embodiment with one or more element from any one or more other embodiment herein, and/or substituting one or more element from one embodiment with one or more element from one or more other embodiment herein.

It is understood, therefore, that this invention is not limited to the particular embodiments disclosed, but is intended to cover all modifications which are within the spirit and scope of the invention as defined by the appended claims; the drawings and/or the above description.

What is claimed is:

1. A method of treating a patient comprising:
administering a PEG based hydrogel to a patient in need thereof to at least one site of administration, the at least one site of administration selected from the group consisting of spinal cord pia mater of the patient, arachnoid mater of the patient, intrathecal portions of spinal nerves of the patient, and directly to spinal cord parenchyma of the patient.

2. The method of claim 1, wherein the step of administering includes applying a composition comprising precursors of the PEG based hydrogel at the at least one site of administration and the precursors react to form the PEG based hydrogel in situ.

3. The method of claim 2, wherein the precursors include a donor and an acceptor and the reaction to form the PEG based hydrogel is a step growth, base-catalyzed reaction between the donor and the acceptor, the donor having a nucleophilic functional group and the acceptor having an electrophilic functional group.

4. The method of claim 3, wherein the nucleophilic functional group is a thiol and the electrophilic functional group is an acrylate.

5. The method of claim 3, wherein the donor is a trifunctional thiol polymer and the acceptor is a bifunctional acrylate polymer.

6. The method of claim 3, wherein the donor is ethoxylated trimethylolpropane tri-3-mercaptopropionate and the acceptor is poly(ethylene glycol) diacrylate.

7. The method of claim 6, wherein the ethoxylated trimethylolpropane tri-3-mercaptopropionate is added at a concentration of 40 weight percent polymer.

8. The method of claim 6, wherein the poly(ethylene glycol) diacrylate has an average Mn of ˜575 g/mol-1100 g/mol.

9. The method of claim 6, wherein the poly(ethylene glycol) diacrylate has an average Mn of ˜575 g/mol.

10. The method of claim 6, wherein the poly(ethylene glycol) diacrylate has an average Mn of ˜675 g/mol-725 g/mol.

11. The method of claim 6, wherein the poly(ethylene glycol) diacrylate has an average Mn of ˜900 g/mol-1100 g/mol.

12. The method of claim 2, wherein the PEG based hydrogel further comprises at least one bioactive epitope.

13. The method of claim 12, wherein the at least one bioactive epitope includes one or more of a peptide, a protein, an antibody, or an aptamer.

14. The method of claim 13, wherein the peptide is selected from the group consisting of RGD and IKVAV.

15. The method of claim 2, wherein the step of forming the PEG based hydrogel occurs in an isotonic buffer that has a salt ion concentration modeled on cerebral spinal fluid.

16. The method of claim 15, wherein the isotonic buffer has a pH between 7.2-7.3.

17. The method of claim 15, wherein the isotonic buffer has an osmolarity between 270-310 mOsm/kg as measured by freezing point depression osmometry.

18. The method of claim 15, wherein the salt ion concentration is artificial cerebral spinal fluid comprising 149 mM sodium chloride (NaCl), 3 mM potassium chloride (KCl), 1.4 mM calcium chloride dihydrate (CaCl₂.2H₂O), 0.8 mM magnesium chloride hexahydrate (MgCl₂.6H₂O), 0.8 mM sodium phosphate dibasic (Na₂HPO₄), and 0.2 mM sodium phosphate monobasic (NaH₂PO₄).

19. The method of claim 2, wherein the composition includes at least one additional agent.

20. The method of claim 19, wherein the at least one additional agent is selected from the group consisting of therapeutic agents, a corticosteroid, methylprednisolone, an anti-inflammatory drug, an anti-CD11d antibody, an angiogenesis promoting growth factor, VEGF, PDGF, decorin, chondroitinase ABC, an anti-Nogo-A antibody, recombinant BA-210 protein, an agent that can alleviate pain, morphine, clonidine, gabapentin, bupivicane, ziconotide, and baclofen.

21. The method of claim 2 further comprising applying at least one additional agent at the at least one site of administration.

22. The method of claim 21, wherein the at least one additional agent is selected from the group consisting of therapeutic agents, a corticosteroid, methylprednisolone, an anti-inflammatory drug, an anti-CD11d antibody, an angiogenesis promoting growth factor, VEGF, PDGF, decorin, chondroitinase ABC, an anti-Nogo-A antibody, recombinant BA-210 protein, an agent that can alleviate pain, morphine, clonidine, gabapentin, bupivicane, ziconotide, and baclofen.

23. The method of claim 21, wherein the step of applying the at least one additional agent occurs at one of before, during, or after the step of applying the composition.

24. A composition comprising a PEG based hydrogel comprising an aqueous solvent and formed by reaction of a donor and an acceptor via a step growth, base-catalyzed reaction between the donor and the acceptor, the donor having a nucleophilic functional group and the acceptor having an electrophilic functional group.

25. The composition of claim 24, wherein the nucleophilic functional group is a thiol and the electrophilic functional group is an acrylate.

26. The composition of claim 24, wherein the donor is a trifunctional thiol polymer and the acceptor is a bifunctional acrylate polymer.

27. The composition of claim 24, wherein the donor is ethoxylated trimethylolpropane tri-3-mercaptopropionate and the acceptor is poly(ethylene glycol) diacrylate.

28. The composition of claim 27, wherein the ethoxylated trimethylolpropane tri-3-mercaptopropionate is at a concentration of 40 weight percent polymer.

29. The composition of claim 27, wherein the poly(ethylene glycol) diacrylate has an average Mn of ˜575 g/mol-1100 g/mol.

30. The composition of claim 27, wherein the poly(ethylene glycol) diacrylate has an average Mn of ˜575 g/mol.

31. The composition of claim 27, wherein the poly(ethylene glycol) diacrylate has an average Mn of ˜675 g/mol-725 g/mol.

32. The composition of claim 27, wherein the poly(ethylene glycol) diacrylate has an average Mn of ˜900 g/mol-1100 g/mol.

33. The composition of claim 24 further comprising at least one bioactive epitope covalently bound to the PEG based hydrogel.

34. The composition of claim 33, wherein the at least one bioactive epitope includes one or more of a peptide, a protein, an antibody, or an aptamer.

35. The composition of claim 34, wherein the peptide is selected from the group consisting of RGD and IKVAV.

36. The composition of claim 24, wherein the aqueous solvent is an isotonic buffer that has a salt ion concentration modeled on cerebral spinal fluid.

37. The composition of claim 36, wherein the isotonic buffer has a pH between 7.2-7.3.

38. The composition of claim 36, wherein the isotonic buffer has an osmolarity between 270-310 mOsm/kg as measured by freezing point depression osmometry.

39. The composition of claim 36, wherein the salt ion concentration is artificial cerebral spinal fluid comprising 149 mM sodium chloride (NaCl), 3 mM potassium chloride (KCl), 1.4 mM calcium chloride dihydrate (CaCl₂.2H₂O), 0.8 mM magnesium chloride hexahydrate (MgCl₂.6H₂O), 0.8 mM sodium phosphate dibasic (Na₂HPO₄), and 0.2 mM sodium phosphate monobasic (NaH₂PO₄).

40. The composition of claim 24, wherein the composition includes at least one additional agent.

41. The composition of claim 40, wherein the at least one additional agent is selected from the group consisting of therapeutic agents, a corticosteroid, methylprednisolone, an anti-inflammatory drug, an anti-CD11d antibody, an angiogenesis promoting growth factor, VEGF, PDGF, decorin, chondroitinase ABC, an anti-Nogo-A antibody, recombinant BA-210 protein, an agent that can alleviate pain, morphine, clonidine, gabapentin, bupivicane, ziconotide, and baclofen.
